(19) United States
(12) Patent Application Publication
(10) Pub. No.: US 2002/0177612 A1
(43) Pub. Date: Nov. 28, 2002

(54) Composition comprising 5-[4-[2-(N-methyl-N-2-pyridy)amino)ethoxy]benzyl]thiazolidine-2,4-dione

(75) Inventors: Jeffrey Roger Granett, Rosemont, PA (US); Hamish Ross, Berwyn, PA (US); Jaikrishna Patel, (US); Robin Kevin John Price, Great Dunmow (GB); Paul Nigel Wray, Crawley (GB)

Correspondence Address: GLAXOSMITHKLINE, Corporate Intellectual Property - UW2220, P.O. Box 1539, King of Prussia, PA, 19406-0939, US

(73) Assignee: SmithKline Beecham p.I.c.

(21) Appl. No.: 10/068,514

(22) Filed: Feb. 5, 2002

Related U.S. Application Data
(63) Continuation of application No. 09/424,278, filed on Mar. 21, 2000, now abandoned.
Application No. 09/424,278 is a 371 of international application No. PCT/EP98/03478, filed on Jun. 2, 1998.

(30) Foreign Application Priority Data
Jun. 5, 1997 (GB) 9711683.4
Jun. 18, 1997 (GB) 9712851.6

(51) Int. Cl. A61K 31/4439

(52) U.S. Cl. 514/340

(57) ABSTRACT

A pharmaceutical composition comprising Compound (1), characterised in that the composition comprises 2 to 12 mg of Compound (1) in a pharmaceutically acceptable form and optionally a pharmaceutically acceptable carrier therefor, the use of such a composition in medicine, processes for the preparation of such a composition and intermediate composition useful in such a process.

This invention relates to a composition, in particular to a pharmaceutical composition, and to the use of such a composition in medicine, to processes for the preparation of such a composition and to a composition useful in such a process.

European Patent Application, Publication Number 0,306,228 relates to certain thiazolidinedione derivatives disclosed as having hypoglycaemic and hypolipidaemic activity. One particular thiazolidinedione disclosed in EP 0306228 is 5-[4-[2-(N-methyl-N-(2-pyridyl)amino)ethoxy]benzyl]thiazolidine-2,4-dione (hereinafter ‘Compound (I)’). International Patent Application, publication number W094/05659 discloses certain salts of Compound (I), including the maleate salt at Example 1 thereof.

It is now surprisingly indicated that a discrete and particular daily dosage of Compound (I) provides an especially beneficial effect on glycaemic control and is therefore particularly useful for treatment of diabetes mellitus, especially Type II diabetes and conditions associated with diabetes mellitus.

We have also discovered a new and advantageous method for preparing pharmaceutical compositions, especially unit dosage compositions, containing Compound (I). The new method involves the preparation of a pre-administration concentrate of Compound (I) which thereafter is formulated into the required unit dose in an efficient and economical manner. The new process is particularly advantageous for the preparation of tablets of Compound (I).

Accordingly, in a first aspect the present invention provides a pharmaceutical composition, suitably in unit dosage form, comprising Compound (I), characterised in that the composition comprises 2 to 12 mg of Compound (I) in a pharmaceutically acceptable form and optionally a pharmaceutically acceptable carrier therefor.

Suitable pharmaceutically acceptable forms of Compound (I) include pharmaceutically acceptable salted forms and pharmaceutically acceptable solvated forms, including pharmaceutically acceptable solvated forms of pharmaceutically acceptable salts.

Suitable compositions comprise 2, 3, 4, 5, 6, 7, 8, 9, 10, 11 or 12 mg of Compound (I) in a pharmaceutically acceptable form.

Particular compositions comprise 2 to 4 mg of Compound (I) in a pharmaceutically acceptable form.

Particular compositions comprise 4 to 8 mg of Compound (I) in a pharmaceutically acceptable form.

Particular compositions comprise 8 to 12 mg of Compound (I) in a pharmaceutically acceptable form.

One composition comprises 2 mg of Compound (I) in a pharmaceutically acceptable form.

Preferred compositions comprise 4 mg of Compound (I) in a pharmaceutically acceptable form.

Preferred compositions comprise 8 mg of Compound (I) in a pharmaceutically acceptable form.

Suitable pharmaceutically acceptable salted forms of Compound (I) include those described in EP 0306228 and W094/05659. A preferred pharmaceutically acceptable salt is a maleate.

Suitable pharmaceutically acceptable solvated forms of Compound (I) include those described in EP 0306228 and W094/05659, in particular hydrates.

Compound (I) or a pharmaceutically acceptable salt thereof, or a pharmaceutically acceptable solvate thereof, may be prepared using known methods, for example those disclosed in EP 0306228 and W094/05659. The disclosures of EP 0306228 and W094/05659 are incorporated herein by reference.

Compound (I) may exist in one of several tautomeric forms, all of which are encompassed by the term ‘Compound (I)’ as individual tautomeric forms or as mixtures thereof.

Compound (I) contains a chiral carbon atom, and hence can exist in up to two stereoisomeric forms, the term Compound (I) encompasses all of these isomeric forms whether as individual isomers or as mixtures of isomers, including racemates.

When used herein the term ‘conditions associated with diabetes’ includes those conditions associated with the pre-diabetic state, conditions associated with diabetes mellitus itself and complications associated with diabetes mellitus.

When used herein the term ‘conditions associated with the pre-diabetic state’ includes conditions such as insulin resistance, including hereditary insulin resistance, impaired glucose tolerance and hyperinsulinaemia.

‘Conditions associated with diabetes mellitus itself’ include hyperglycaemia insulin resistance, including acquired insulin resistance and obesity. Further conditions associated with diabetes mellitus itself include hypertension, cardiovascular disease, especially atherosclerosis, certain eating disorders, in particular those requiring the regulation of appetite and food intake, such as disorders associated with under-eating, for example anorexia nervosa and disorders associated with over-eating, for example obesity and anorexia bulimia. Additional conditions associated with diabetes mellitus itself include polycystic ovarian syndrome and steroid induced insulin resistance and gestational diabetes.

‘Complications associated with diabetes mellitus’ includes renal disease, especially renal disease associated with Type II diabetes, including diabetic nephropathy, glomerulonephritis, glomerular sclerosis, nephrotic syndrome, hypertensive nephrosclerosis and end stage renal disease.

As used herein the term ‘pharmaceutically acceptable’ embraces both human and veterinary use: for example the term ‘pharmaceutically acceptable’ embraces a veterinarily acceptable compound.

As used herein the term concentrate with respect to Compound (I) in a pharmaceutically acceptable form means a proportionate amount of Compound (I) in a pharmaceutically acceptable form greater than that present in an administerable composition.

For the avoidance of doubt, when reference is made herein to scalar amounts, including mg amounts and % weight amounts, of ‘Compound (I) in a pharmaceutically acceptable form’, the scalar amount referred to is made in respect of Compound (I) per se: For example 2 mg of Compound (I) in the form of the maleate salt is that amount of maleate salt which contains 2 mg of Compound (I).

Diabetes mellitus is preferably Type II diabetes.

In a further aspect, the invention provides a process for preparing a pharmaceutical composition comprising 2 to 12 mg of Compound (I) in a pharmaceutically acceptable form, and a pharmaceutically acceptable carrier therefor, which process comprises admixing 2 to 12 mg of Compound (I) in a pharmaceutically acceptable form and the pharmaceutically acceptable carrier and optionally thereafter formulating the composition produced into an administerable form.

As indicated above the invention also provides a further process for preparing a pharmaceutical composition comprising Compound (I) in a pharmaceutically acceptable form which is particularly suitable for preparing a range of unit dosage forms of Compound (I). Accordingly, the invention further provides a process for preparing a pharmaceutical composition of Compound (I) in a pharmaceutically acceptable form and a pharmaceutically acceptable carrier, which process comprises:

(i) preparing a first composition comprising Compound (I) in a pharmaceutically acceptable form and a first pharmaceutically acceptable carrier;

(ii) admixing the first composition with a second pharmaceutically acceptable carrier to provide the required composition of Compound (I) and optionally thereafter formulating the composition produced into an administerable form.

A preferred administerable form of the pharmaceutical composition of Compound (I) is a unit dose composition.

Unless otherwise specified, suitable unit doses comprise up to 12 mg, such as 1 to 12 mg, of Compound (I) in a pharmaceutically acceptable form.

Other unit doses include those mentioned herein.

A key component of the last above mentioned process is the first composition. Accordingly, the present invention also provides a composition for use as a first composition in a process for preparing a unit dose of Compound (I) in a pharmaceutically acceptable form.

The invention also provides a composition comprising Compound (I) in a pharmaceutically acceptable form and optionally a pharmaceutically acceptable carrier, characterised in that the composition is a pharmaceutically acceptable, pre-administration composition.

A suitable pharmaceutically acceptable, pre-administration composition is a concentrate, preferably a granular concentrate, of Compound (I) in a pharmaceutically acceptable form. The granular concentrate is particularly well adapted to be diluted to provide a composition for administration, preferably a tablet.

In a further aspect the invention provides a composition comprising Compound (I) in a pharmaceutically acceptable form and a pharmaceutically acceptable carrier, characterised in that the composition is a concentrate of Compound (I) in a pharmaceutically acceptable form, adapted to be diluted so as to provide a composition for administration.

Suitably, the first composition, pre-administration composition or dilutable composition (hereinafter referred to for convenience as ‘the first composition’) contains up to 50% by weight, for example an amount in the range of from 2 to 50% by weight, of Compound (I) in a pharmaceutically acceptable form.

Favourably, the first composition contains an amount of Compound (I) in a pharmaceutically acceptable form in the range of from 5 to 20% by weight, in particular 5%, 10% or 15% by weight, for example 10% by weight.

The processes of the invention can provide pharmaceutical compositions of Compound (I) in any conventionally administerable form, including orally or parenterally adminsterable forms. They are particularly well adapted for preparing orally administrable forms, especially tablet forms of Compound (I) in a pharmaceutically acceptable form.

The first pharmaceutically acceptable carrier can comprise any conventional pharmaceutically acceptable carrier comprising conventional pharmaceutically acceptable excipients, including those disclosed in the reference texts mentioned below. However, as it is not essential that the first pharmaceutically acceptable carrier is in an administerable form, then it need not contain excipients solely associated with administration. For example the first pharmaceutically acceptable carrier need not contain a lubricant.

The second pharmaceutically acceptable carrier includes any conventional pharmaceutically acceptable carrier comprising any conventional pharmaceutically acceptable excipient, including disintegrants, diluents and lubricants, including those disclosed in the reference texts mentioned below.

One particular first composition comprises Compound (I) in a pharmaceutically acceptable form, a disintegrant, a binder and a diluent.

A suitable disintegrant is sodium starch glycollate.

A suitable binder is a methyl cellulose binder, such as hydroxypropyl methylcellulose 2910.

Suitable diluents include cellulose, for example a microcrystalline cellulose, and lactose monohydrate.

A suitable lubricant is magnesium stearate.

We have found that a particularly advantageous first composition contains Compound (I) in a pharmaceutically acceptable form, sodium starch glycollate, hydroxypropyl methylcellulose 2910, microcrystalline cellulose and lactose monohydrate, especially when in a granular form. This granular form has been found to be particularly stable.

When the first composition contains about 10% by weight of Compound (I) in a pharmaceutically acceptable form, it is readily dilutable to give unit dose compositions comprising in the range of between 2 to 12 mg, especially 2 to 8 mg, 2 to 4 mg, 4 to 8 mg and 8 to 12 mg of Compound (I) in a pharmaceutically acceptable form.

The preparation of the first composition is suitably carried using any conventional method appropriate to the nature of the said first composition, for example wet granulation methods provide the first composition in granular form.

Methods for formulating the compositions of the invention into administerable forms include conventional formulation methods as disclosed in the reference texts cited herein, including tabletting methods.

The administerable compositions of the invention are preferably adapted for oral administration. However, they may also be adapted for other modes of administration, for example parenteral administration, sublingual or transdermal administration.

The administerable compositions may be in the form of tablets, capsules, powders, granules, lozenges, suppositories, reconstitutable powders, or liquid preparations, such as oral or sterile parenteral solutions or suspensions.

In order to obtain consistency of administration it is preferred that a composition of the invention is in the form of a unit dose.

Unit dose presentation forms for oral administration may be tablets and capsules and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulphate.

Unless otherwise prescribed, compositions of the invention are preferably in unit dosage form in an amount appropriate for the relevant daily dosage, suitable unit dosages comprise 1, 2, 3, 4, 5, 6, 7, 8, 9, 10, 11 or 12 mg of Compound (I) in a pharmaceutically acceptable form.

The solid compositions for example the oral compositions may be prepared by conventional methods of blending, filling or tabletting. As required repeated blending operations may be used to distribute the active agent throughout those compositions employing large quantities of fillers. Such operations are of course conventional in the art. The tablets may be coated according to methods well known in normal pharmaceutical practice, in particular with an aqueous film coating.

Liquid compositions, for example oral liquid compositions, may be in the form of emulsions, syrups, or elixirs, or they may be in a dry product form to be reconstituted with water or other suitable vehicle before use. Such liquid preparations may contain conventional additives such as suspending agents, for example sorbitol, syrup, methyl cellulose, gelatin, hydroxyethylcellulose, carboxymethylcellulose, aluminium stearate gel, hydrogenated edible fats; emulsifying agents, for example lecithin, sorbitan monooleate, or acacia; non-aqueous vehicles (which may include edible oils), for example almond oil, fractionated coconut oil, oily esters such as esters of glycerine, propylene glycol, or ethyl alcohol; preservatives, for example methyl or propyl p-hydroxybenzoate or sorbic acid; and if desired conventional flavouring or colouring agents.

Parenteral compositions, including parenteral administration compositions for example unit dosage compositions, comprise the active compound and a sterile vehicle, and, depending upon the concentration used, can be either suspended or dissolved in the vehicle. In preparing solutions for parenteral administration the composition of the invention may be dissolved in water for injection and filter sterilized before filling into a suitable vial or ampoule and sealing. Advantageously, adjuvants such as a local anaesthetic, a preservative and buffering agents can be dissolved in the vehicle. To enhance the stability, the composition can be frozen after filling into the vial and the water removed under vacuum. Parenteral suspensions are prepared in substantially the same manner, except that the active compound is suspended in the vehicle instead of being dissolved, and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

Unless otherwise specified the compositions of the invention may contain from 0.1% to 99% by weight, preferably from 10-60% by weight, of the active material, depending upon the method of administration.

The composition may, if desired, be in the form of a pack accompanied by written or printed instructions for use.

The compositions of the invention may be prepared and formulated according to conventional methods, such as those disclosed in standard reference texts, for example the British and US Pharmacopoeias, Remington's Pharmaceutical Sciences (Mack Publishing Co.), Martindale The Extra Pharmacopoeia (London, The Pharmaceutical Press) and Harry's Cosmeticology (Leonard Hill Books).

The present invention also provides a pharmaceutical composition comprising 2 to 12 mg of Compound (I) and a pharmaceutically acceptable carrier therefor, for use as an active therapeutic substance.

In particular, the present invention provides a pharmaceutical composition comprising 2 to 12 mg of Compound (I) in a pharmaceutically acceptable form, for use in the treatment of diabetes mellitus, especially Type II diabetes, and conditions associated with diabetes mellitus.

The composition of the invention may be administered from 1 to 6 times a day, but most preferably 1 or 2 times per day.

Accordingly, in a further aspect the invention provides a method for treatment of diabetes mellitus, especially Type II diabetes and conditions associated with diabetes mellitus, in a mammal such as a human, which method comprises administering per day 2 to 12 mg of Compound (I) in a pharmaceutically acceptable form, to a mammal in need thereof.

Particularly, the method comprises the administration of 2 to 4 , 4 to 8 or 8 to 12 mg of Compound (I) in a pharmaceutically acceptable form.

Particular dosages are 2 mg/day, 4 mg/day, including 2 mg twice per day, and 8 mg/day, including 4 mg twice per day.

Particularly, the method comprises the administration of 2 to 4 mg of Compound (I) in a pharmaceutically acceptable form.

Particularly, the method comprises the administration of 4 to 8 mg of Compound (I) in a pharmaceutically acceptable form.

Particularly, the method comprises the administration of 8 to 12 mg of Compound (I) in a pharmaceutically acceptable form.

Preferably, the method comprises the administration of 2 mg of Compound (I) in a pharmaceutically acceptable form.

Preferably, the method comprises the administration of 4 mg of Compound (I) in a pharmaceutically acceptable form.

Preferably, the method comprises the administration of 8 mg of Compound (I) in a pharmaceutically acceptable form.

A range of 2 to 4 mg includes a range of 2.1 to 4, 2.2 to 4, 2.3 to 4, 2.4 to 4, 2.5 to 4, 2.6 to 4, 2.7 to 4, 2.8 to 4, 2.9 to 4 or 3 to 4 mg.

A range of 4 to 8 mg includes a range of 4.1 to 8, 4.2 to 8, 4.3 to 8, 4.4 to 8,4.5 to 8, 4.6 to 8, 4.7 to 8, 4.8 to 8, 4.9 to 8, 5 to 8,6to 8 or 7 to 8 mg.

A range of 8 to 12 mg includes a range of 8.1 to 12, 8.2 to 12, 8.3 to 12, 8.4 to 12, 8.5 to 12, 8.6 to 12, 8.7 to 12, 8.8 to 12, 8.9 to 12, 9to 12, 10to 12or 11 to 12mg.

No adverse toxicological effects have been established for the compositions or methods of the invention in the abovementioned dosage ranges.

The following examples illustrate the invention but do not limit it in any way.

### EXAMPLE 1

### Concentrate Preparation

Approximately two thirds of the lactose monohydrate is passed through a suitable screen and blended with the milled maleate salt of Compound (I).

Sodium starch glycollate, hydoxypropyl methylcellulose, microcrystalline cellulose and the remaining lactose are passed through a suitable screen and added to the mixture. Blending is then continued. The resulting mixture is then wet granulated with purified water. The wet granules are then screened, dried on a fluid bed drier and the dried granules are passed through a further screen and finally homogenised.

% COMPOSITION OF GRANULAR CONCENTRATE

| Ingredient | Quantity (%) |
|---|---|
| Milled Compound (I) as maleate | 13.25 (pure |
| salt | maleate salt) |
| Sodium Starch Glycollate | 5.00 |
| Hydoxypropyl Methylcellulose | 5.00 |
| 2910 | |
| Microcrystalline Cellulose | 20.0 |
| Lactose Monohydrate, regular | to 100 |
| grade | |
| Purified water | * |

*Removed during processing.

### EXAMPLE 2

### Formulation of the Concentrate into Tablets.

The granules from Example 1 are placed into a tumble blender. Approximately two thirds of the lactose is screened and added to the blender. The microcrystalline cellulose, sodium starch glycollate, magnesium stearate and remaining lactose are screened and added to the blender and the mixture blended together. The resulting mix is then compressed on a rotary tablet press to a target weight of 150 mg for the 1, 2 and 4 mg tablets and to a target weight of 300 mg for the 8 mg tablets.

The tablet cores are then transferred to a tablet coating machine, pre-warned with warm air (approximately 65° C.) and film coated until the tablet weight has increased by 2.0% to 3.5%.

| | Quantity (mg per Tablet) | | | |
|---|---|---|---|---|
| Tablet Strength | 1.0 mg | 2.0 mg | 4.0 mg | 8.0 mg |
| Active Ingredient: | | | | |
| Compound (I) maleate Concentrate | 10.00 | 20.00 | 40.00 | 80.00 |
| granules | | | | |
| Other Ingredients: | | | | |
| Sodium Starch Glycollate | 6.96 | 6.46 | 5.46 | 10.92 |
| Microcrystalline Cellulose | 27.85 | 25.85 | 21.85 | 43.70 |
| Lactose monohydrate | 104.44 | 96.94 | 81.94 | 163.88 |
| Magnesium Stearate | 0.75 | 0.75 | 0.75 | 1.50 |
| Total Weight of Tablet Core | 150.0 | 150.0 | 150.0 | 300.0 |
| Aqueous film coating material | 4.5 | 4.5 | 4.5 | 9.0 |
| Total Weight of Film Coated | 154.5 | 154.5 | 154.5 | 309.0 |
| Tablet | | | | |

1. A pharmaceutical composition comprising 5-[4-[2-(N-methyl -N-(2-pyridyl) amino)ethoxy]benzyl]thiazolidine-2,4-dione (hereinafter ‘Compound (I), characterised in that the composition comprises 2 to 12 mg of Compound (1) in a pharmaceutically acceptable form and optionally a pharmaceutically acceptable carrier therefor.

22. A composition according to claim 1 which comprises 2 to 4 mg of Compound (I) in a pharmaceutically acceptable form.

23. A composition according to claim 1 which comprises 4 to 8 mg of Compound (I) in a pharmaceutically acceptable form.

24. A composition according to claim 1 which comprises 8 to 12 mg of Compound (I) in a pharmaceutically acceptable form.

25. A composition according to claim 1 which comprises 2 mg of Compound (J) in a pharmaceutically acceptable form.

26. A composition according to claim 1 which comprises 4 mg of Compound (I) in a pharmaceutically acceptable form.

27. A composition according to claim 1 which comprises 8 mg of Compound (I) in a pharmaceutically acceptable form.

28. A composition according to claim 1 which comprises the maleate salt of Compound (I)

29. A process for preparing a pharmaceutical composition comprising 2 to 12 mg of 5-[4-[2-(N-methyl-N-(2-pyridyl)amino)ethoxy]benzyl]thiazolidine-2,4-dione in a pharmaceutically acceptable form and a pharmaceutically acceptable carrier therefor, which process comprises admixing 2 to 12 mg of the dione in a pharmaceutically acceptable form and the pharmaceutically acceptable carrier.

30. A process according to claim 29 wherein the composition is in unit dosage form.

31. A process according to claim 29 wherein the composition is a tablet.

32. A process for preparing a pharmaceutical composition of 5-[4-[2-(N-methyl-N-(2-pyridyl) amino)ethoxy]benzyl]thiazolidine-2,4-dione in a pharmaceutically acceptable form and a pharmaceutically acceptable carrier, which process comprises:
(i) preparing a first composition comprising the dione in a pharmaceutically acceptable form and a first pharmaceutically acceptable carrier;
(ii) admixing the first composition with a second pharmaceutically acceptable carrier to provide the required composition of dione and optionally thereafter formulating the composition produced into an administerable form.

33. A process according to claim 32 wherein the composition is in unit dosage form.

34. A process according to claim 32 wherein the composition is a tablet.

35. A composition comprising 5-[4-[2-(N-methyl-N-(2-pyridyl) amino)ethoxy]benzyl]thiazolidine-2,4-dione in a pharmaceutically acceptable form and optionally a pharmaceutically acceptable carrier, wherein the composition is a pharmaceutically acceptable, pre-administration composition.

36. A pre-administration composition according to claim 35 which is a concentrate of the dione in a pharmaceutically acceptable form.

37. A composition according to claim 35 adapted to be diluted with a pharmaceutically acceptable diluent so as to provide a composition for administration.

38. A composition according to claim 35 which contains up to 50% by weight of the dione in a pharmaceutically acceptable form.

39. A composition according to claim 35 which contains an amount of the dione in a pharmaceutically acceptable form in the range of from 5 to 20% by weight.

40. A composition according to claim 35 which contains 5%, 10% or 15% by weight of the dione in a pharmaceutically acceptable form.

41. A composition according to claim 35, which contains Compound (I) in a pharmaceutically acceptable form, sodium starch glycollate, hydroxypropyl methylcellulose 2910, microcrystalline cellulose and lactose monohydrate.

42. A composition according to claim 35 in granular form.
